(19) United States
(12) Patent Application Publication
(10) Pub. No.: US 2010/0086925 A1
(43) Pub. Date: Apr. 8, 2010

(54) MICROFLUIDIC STRUCTURE FOR MULTI-ASSAY AND MICROFLUIDIC DEVICE COMPRISING THE SAME

(75) Inventors: Jung Nam LEE, Incheon (KR); Beom Seok LEE, Hwaseong-si (KR); Kui Hyun KIM, Suwon-si (KR); Ji Won KIM, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD., Suwon-si (KR)

(21) Appl. No.: 12/494,383

(22) Filed: Jun. 30, 2009

(30) Foreign Application Priority Data
Oct. 2, 2008 (KR) 10-2008-0097405

(51) Int. Cl. C12Q 1/68; C12M 1/34; G01N 33/53

(52) U.S. Cl. 435/6; 435/287.2; 435/7.5

(57) ABSTRACT

Exemplary embodiments relate to a microfluidic structure including: a plurality of sample chambers; a reaction chamber in which at least two types of materials, which respectively specifically react with at least two types of target materials, are immobilized; a detection chamber connected to the reaction chamber; a path connecting the chambers; and a valve for opening and closing the path, and a microfluidic device including the microfluidic structure. Since at least two types of materials specifically binding to target materials are immobilized in a reaction chamber of the microfluidic structure, space may be efficiently used and the target materials may be assayed in a one-step test. An internal space of the microfluidic device using the microfluidic structure, the amount of samples, and costs for manufacturing the microfluidic device may be reduced, and internal quality control may be efficiently performed using the microfluidic structure as a control for the operations.

### CROSS-REFERENCE TO RELATED APPLICATIONS

This application claims priority to Korean Patent Application No. 10-2008-0097405 filed on Oct. 2, 2008, and all the benefits accruing therefrom under 35 U.S.C. §119, the content of which in its entirety is herein incorporated by reference.

### BACKGROUND

1. Field

One or more embodiments relate to a microfluidic structure for a multi-assay and a microfluidic device including the same, and more particularly, to a microfluidic structure including: a plurality of sample chambers; a reaction chamber in which at least two types of materials, which respectively specifically react with at least two types of target materials, are immobilized; a detection chamber connected to the reaction chamber; a path connecting the chambers; and a valve for opening and closing the path, and a microfluidic device based on a centrifugal force, including the microfluidic structure.

2. Description of the Related Art

In general, a driving pressure is necessary in order to transport a fluid in a microfluidic structure included in a microfluidic device. The driving pressure may be capillary pressure or pressure supplied by an additional pump. Recently, a microfluidic device having a microfluidic structure on a disc-shaped platform, which performs a series of operations using a centrifugal force, i.e., lab-on-a disc or Lab CD, has been proposed as a clinical diagnosis and assay device designed to inexpensively and easily detect a small amount of target materials in a fluid.

The lab-on-a disc which stands for ‘laboratory on a disc’ is a device in which various units adapted for analysis of bio-molecules are integrated on a disc-shaped device. When a bio-sample such as blood is introduced into a microfluidic structure formed on the disc, a fluid such as the sample or a reagent may be transported using a centrifugal force without using an additional driving system, such as a driving pressure, to transport the fluid.

Thus, there is a need to develop a device including a plurality of chambers in order to efficiently analyze a variety of bio-samples using the disc-shaped assay device.

### SUMMARY

One or more embodiments include a microfluidic structure by which at least two types of target materials may be detected in a single reaction chamber. One or more embodiments also include a microfluidic device based on a centrifugal force, which includes a rotation body and the microfluidic structure. One or more embodiments also include a method of assaying at least two types of target materials by using the microfluidic device. One or more embodiments also include a method of internal quality control by using the microfluidic device.

Additional aspects will be set forth in part in the description which follows and, in part, will be apparent from the description, or may be learned by practice of the invention.

Inventors found that a plurality of materials may be detected and assayed in a single reaction chamber if at least two types of materials, which respectively specifically react with at least two types of target materials, are immobilized in the reaction chamber. In addition, one of the reaction results may be used as a control for internal quality control since each of the reactions with the target materials does not affect the others.

To achieve the above and/or other aspects, one or more embodiments may include a microfluidic structure including: a plurality of sample chambers; a reaction chamber in which at least two types of materials (“capture material(s)”), which respectively specifically react with at least two types of target materials, are immobilized; a detection chamber connected to the reaction chamber; a path connecting the chambers; and a valve for opening and closing the path.

The at least two types of capture materials, which respectively specifically react with the at least two types of target materials, may be different from each other.

The target materials and the capture materials, which respectively specifically react with the target materials, may be selected from the group consisting of a protein, an antigen, an antibody, an enzyme, deoxyribonucleic acid (DNA), peptide nucleic acid (PNA), ribonucleic acid (RNA), a hormone, and a chemical material. The target materials and the capture materials may be selected from the group consisting of an antigen, an antibody, and a protein.

The plurality of sample chambers may include at least one chamber selected from the group consisting of a buffer solution chamber, a substrate solution chamber, a probe solution chamber, and a biological sample chamber including at least two types of target materials.

The probe solution may include at least two types of detector probes which respectively specifically react with each of the at least two types of target materials. The at least two types of detector probes may be respectively combined with different markers, and have activity which is the same as or different from that of the materials immobilized in the reaction chamber. The marker may be one selected from the group consisting of an enzyme, a fluorescent material, a radioactive isotope, and a chemical material.

To achieve the above and/or other aspects, one or more embodiments may include a microfluidic device based on centrifugal force including a rotation body and the microfluidic structure, wherein a fluid in the microfluidic structure is transported using centrifugal force generated by the rotation of the rotation body.

To achieve the above and/or other aspects, one or more embodiments may include a method of assaying at least two types of target materials using the microfluidic device, the method including: adding a sample including at least two types of target materials to a reaction chamber so that the at least two types of target materials contact with at least two types of materials which respectively specifically react with the at least two types of target materials; and adding a solution including at least two types of detector probes, which respectively specifically react with the at least two types of target materials and are combined with markers, to the reaction chamber, so that the at least two types of target materials contact with the at least two types of detector probes.

The method may further include detecting target materials by measuring signals from the markers combined with the detector probes. The signal may be a luminescent (e.g., fluorescent and phosphorescent) signal, a radial signal, and an electrical signal. In addition, the signal may be generated from a chromogenic material (i.e., a light-absorbing material) which is a substrate of an enzyme.

The at least two types of capture materials, which respectively specifically react with the at least two types of target materials, may be different materials from each other.

The target materials and the capture materials, which respectively specifically reacting with the target materials, may be selected from the group consisting of protein, an antigen, an antibody, an enzyme, DNA, PNA, RNA, a hormone, and a chemical material. The target materials and the capture materials may be selected from the group consisting of an antigen, an antibody, and protein.

The solution including the detector probes may include at least two types of detector probes, which respectively specifically react with the at least two types of target materials. The at least two types of detector probes may be combined with different markers and have activity which is the same as or different from that of the materials immobilized in the reaction chamber. The marker may be an enzyme, a fluorescent material, a radioactive isotope or a chemical material, and the enzyme may be various enzymes such as horseradish peroxidase (HRP) or alkaline phosphatase (AP).

The marker may be an enzyme, and the detection may be performed by adding a substrate, which is converted into a material absorbing light (i.e., a chromogenic material) at a particular wavelength by the enzyme, to the reaction chamber to form the chromogenic material, and measuring signals from the chromogenic material. The method may further include: adding a first substrate, which is to be converted into a first chromogenic material by a first enzyme, to the reaction chamber to form a first chromogenic material, transporting the first chromogenic material to a first detection chamber, and measuring a first signal from the first chromogenic material; and adding a second substrate, which is to be converted into a second chromogenic material by a second enzyme, to the reaction chamber to form a second chromogenic material, transporting the second chromogenic material to a second detection chamber, and measuring a second signal from the second chromogenic material. The first and the second enzymes may respectively be HRP and AP, and the first and the second substrates may be 3,3 prime, 5,5′-tetramethylbenzidene (TMB), 2,2′-azino-bis-(3-ethylbenzthiazoline-6-sulfonic acid) (ABTS), o-phenylenediamine dihydrochloride (OPD) or 3,3′-Diaminobenzidine (DAB), and 4-methylumbelliferyl phosphate (MUP) or p-nitrophenyl phosphate (PNPP).

The sample and the solution including the detector probes may be added to the reaction chamber simultaneously or sequentially.

The adding the solution including the at least two types of detector probes to the reaction chamber may be performed by sequentially or simultaneously adding a solution including a first detector probe and a solution including a second detector probe to the reaction chamber.

The detection may be performed in the reaction chamber or in the detection chamber to which the reaction solution is transported from the reaction chamber in the detecting the target materials. If the detection is performed in the reaction chamber, the marker may be a luminescent material, a radioactive isotope or a chemical material, and the signal may be measured in the reaction chamber. In this regard, the reaction chamber may be optically transparent. If the detection is performed in the detection chamber to which the reaction solution is transported from the reaction chamber, the reaction solution may be transported to at least one (e.g., at least two) detection chamber.

The method may further include cleaning between processes to remove unreacted materials or materials which can not react.

To achieve the above and/or other aspects, one or more embodiments may include a method assaying at least two types of target materials using the microfluidic device, the method including: adding a sample including at least two types of target materials to a reaction chamber so that the at least two types of target materials contact with capture materials, which respectively specifically react with at least two types of target materials, wherein if one or more the target material is a nucleic acid, the nucleic acid is combined with a marker; and adding a solution including at least two types of detector probes, which respectively specifically react with one of the at least two types of target materials and are combined with a marker, so that the at least two types of target materials contact with the at least two types of detector probes.

If a nucleic acid is the target material, a sandwich method and a direct detection method may be used.

To achieve the above and/or other aspects, one or more embodiments may include a method of controlling internal quality using the microfluidic device, the method including: immobilizing capture materials, which respectively specifically react with a first target material and a second target material, in a reaction chamber; adding the first target material and the second target material to the reaction chamber, wherein the concentration of the second target material is known; detecting the first target material and the second target material by adding a solution including detector probes, which respectively specifically react with the first target material and the second target material and are combined with marker, to the reaction chamber so that the detector probes respectively bind to the first target material and the second target material, and measuring signals from the markers; and evaluating the degree of performance of the operations based on the detection results. The evaluation results of the second target material may be used as a process control.

To achieve the above and/or other aspects, one or more embodiments may include a method of controlling internal quality using the microfluidic device, the method including: immobilizing materials, which respectively specifically react with a first target material and a second target material, in a reaction chamber; adding the first target material and the second target material to the reaction chamber, wherein a concentration of the second target material is known and the second target material is combined with a marker; detecting the first target material by adding a solution including a detector probe, which specifically reacts with the first target material and is combined with a marker, to the reaction chamber so that the detector probe is combined with the first target material, and measuring signals from the marker; and evaluating the degree of performance of the operations based on the detection results.

### BRIEF DESCRIPTION OF THE DRAWINGS

These and/or other aspects will become apparent and more readily appreciated from the following description of the embodiments, taken in conjunction with the accompanying drawings of which:

FIG. 1 is a schematic view of a microfluidic device according to an embodiment;

FIG. 2 schematically shows principles of detecting two types of target materials in a single reaction chamber according to an embodiment, wherein Det1 is a marker conjugated with a first capture material specifically binding to a first target material, and Det2 is a marker conjugated with a second capture material specifically binding to a second target material;

FIG. 3 is a flowchart schematically illustrating a process of detecting at least two types of target materials according to an embodiment;

FIG. 4 is a graph illustrating the results of two types of antigen-antibody reactions according to Example 1; and

FIG. 5 is a graph illustrating the results of Prostate Specific Antigen (PSA) antibody-antigen reaction and interaction between streptavidin and biotin according to Example 2.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

A microfluidic structure according to an embodiment may include: a plurality of sample chambers; a reaction chamber in which at least two types of materials, which respectively specifically react with at least two types of target materials, are immobilized; a detection chamber connected to the reaction chamber; a path connecting the chambers; and a valve for opening and closing the path.

As described above, it is advantageous that a Lab-on-a-Disc may be miniaturized since a fluid such as a sample and a reagent may be transported using a centrifugal force without using an additional driving system. In this regard, research regarding efficient designs of the Lab-on-a-Disc has been conducted in order to quickly assay various target materials using a disc-shaped device in a cost-effective manner. In particular, research regarding the relationship between a sample chamber including various target materials, a reaction chamber in which target materials bind to capture materials, which specifically react with the target materials, and a detection chamber has been conducted. Research regarding the number of reactions, assay time, cost efficiency, space efficiency, and the like has been conducted.

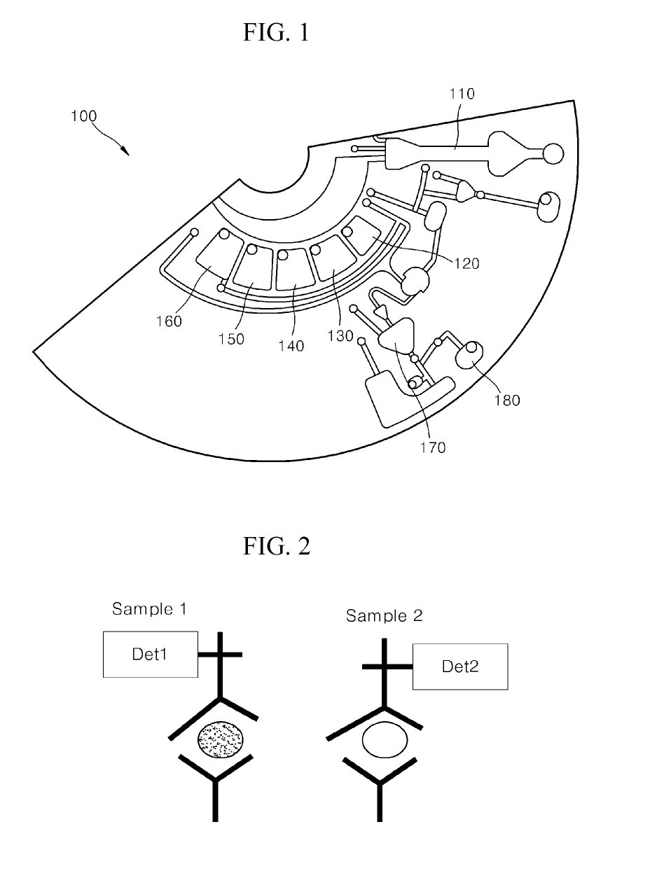

As a result, in a microfluidic device 100 as shown in FIG. 1, since at least two types of capture materials, which respectively specifically react with at least two types of target materials, are immobilized in a single reaction chamber 170, a one-step assay may be performed using a single reaction chamber 170. Thus, an internal space of the disc and the amount of the sample required for the assay may be reduced. That is, there are provided a microfluidic structure in which several reactions may be performed for diagnostic assays based on a centrifugal force since capture materials, which are specifically binding to at least two types of target materials, are immobilized in zones of a single reaction chamber, and a microfluidic device including the microfluidic structure based on centrifugal force. The zones may be a portion or the entire internal surface of the reaction chamber.

FIG. 1 is a schematic view of a microfluidic device according to an embodiment. The microfluidic device may include chambers for storing various buffer solutions, substrates, or probe solutions 140 and 150, and performing various biological or chemical reactions, a storage chamber 110 for storing target materials, a fluid path through which treated fluids and buffer solutions are transported, and a valve for opening and closing the path.

Referring to FIG. 1, a rotation body used in an embodiment may be a disc-shaped platform. The disc-shaped platform may be formed of an acryl or plastic material that has a biologically non-activated surface and is easily formed. However, any material that has chemical and biological stability, optical transparency, and mechanical workability may be used, and the material used to form the platform is not limited.

The rotation body may be formed of a material selected from a group consisting of plastic, polymethyl-methacrylate (PMMA), glass, mica, silica, and a material of silicon wafer. The plastic material may be used due to its low price and high workability. The plastic material may be polypropylene, polyacrylate, poly vinyl alcohol, polyethylene, polymethylmethacrylate, polycarbonate, or the like.

The microfluidic structure including: a plurality of sample chambers; a reaction chamber 170 in which at least two types of materials, which respectively specifically react with at least two types of target materials, are immobilized; a detection chamber 180 connected to the reaction chamber 170; a path connecting the chambers; and a valve for opening and closing the path, may be arranged on the rotation body.

The plurality of sample chambers may include at least one chamber selected from a group consisting of a buffer solution chamber, a substrate solution and/or probe solution chambers 140 and 150, and a biological sample chamber 110 including the target materials.

In this regard, the terminology “target material” may be materials to be assayed from a biological sample, for example, a molecular-level material constituting a living body. Both the target materials and the capture materials that specifically react with the target materials and are immobilized in the reaction chamber, may be biomolecules. For example, the biomolecules may include a protein or peptide, an antigen, an antibody, an enzyme, a deoxyribonucleic acid (DNA), a peptide nucleic acid (PNA), a ribonucleic acid (RNA), a hormone, a chemical material, and the like.

The target materials (e.g., target biomolecules) correspond to analytes, and the materials coated on the inner surface of the reaction chamber are materials that specifically react with the target materials in order to capture the target materials. The coated materials specifically bind to the target materials. Accordingly, a protein-protein interaction, an antigen-antibody reaction, an enzyme-substrate reaction, or the like may be used.

Furthermore, a sequence-specific reaction may be used in nucleic acid-level genetic materials in addition to protein-level materials. That is, nucleic acid molecules may be immobilized in the reaction chamber, and homologous nucleic acid molecules may bind thereto.

The capture materials, which are immobilized in the reaction chamber and respectively specifically react with the target materials, may be different materials from each other. In an exemplary embodiment, the materials may not exhibit cross reactivity with each other.

The “cross reactivity” is a phenomenon that occurs when the capture materials coated on the reaction chamber react with not only the target materials but also materials having a structure similar to or partially the same as that of the target materials. For example, the cross reactivity is a phenomenon whereby an antibody binds to a non-target antigen.

The at least two capture materials coated on the internal surface of the reaction chamber should specifically react with the respective target materials and may not exhibit cross reactivity. For example, in an antigen-antibody reaction employing a first and a second antibody as capture materials, wherein the first capture antibody reacts with a first target antigen in a sample and the second capture antibody reacts with a second target antigen in the sample, the first antigen should not include an epitope which is not a target material of the first capture antibody, but is recognized by the second capture antibody.

Meanwhile, the “probe solution” is a solution used to treat the resultant obtained by binding the target materials and the capture materials in the reaction chamber. The probe solution includes a detector probe. The detector probe may be marked with a marker specifically binding to the target materials of a target material-capture material complex. If the marker is an enzyme, signals from the marker may be detected by adding a chromogenic substrate of the enzyme to the reaction chamber to convert the chromogenic substrate to a chromogenic material by the action of the enzyme, and measuring light signals in the reaction chamber. Alternatively, light signals may be detected from the chromogenic material in the detection chamber by transporting the chromogenic material from the reaction chamber to at least one detection chamber via paths connected to the reaction chamber.

The “detector probe” indicates a material that may detect each of the target materials. Different markers are combined with the detector probes and respectively specifically react with the target materials. Materials used to form the detector probes may have the same or different activities compared with the capture materials.

In addition, the marker combined with the detector probe may be any marker that may detect another material without limitation, and an enzyme, a fluorescent material, a radioactive isotope, or a chemical material.

In particular, various enzymes such as horseradish peroxidase (HRP) or alkaline phosphatase (AP) may be used, or a biological molecule to which fluorescent materials having different wavelengths bind may be used as the detector probe.

Thus, the number of detection chambers may be the same as that of the target materials in the microfluidic structure. Alternatively, a single chamber may be used based on the properties of the marker. That is, if the marker is a fluorescent material, or participates in another reaction by absorbing light at a different wavelength, several materials may be measured in a single chamber by changing the wavelength of the detector. The detection chamber may be the same as or different from the reaction chamber.

Meanwhile, the arrangement of the chambers contained in the microfluidic structure may be designed in the rotation body based on the path of a fluid moved by the centrifugal force. The chambers respectively including each of a buffer solution, a probe solution, and a sample including a target material may be disposed closer to the center of the rotation body, the detection chamber may be disposed farther away from the center of the rotation body, and the reaction chamber may be disposed between the sample chambers and the detection chamber.

An embodiment may provide a method of assaying at least two types of target materials using the microfluidic device, the method including: adding a sample including at least two types of target materials to a reaction chamber so that the at least two types of target materials contact with capture materials, which respectively specifically react with the target materials; and adding a solution including at least two types of detector probes, which respectively specifically react with the at least two types of target materials and are combined with markers so that the target materials contact with the detector probes.

The terminologies are described above.

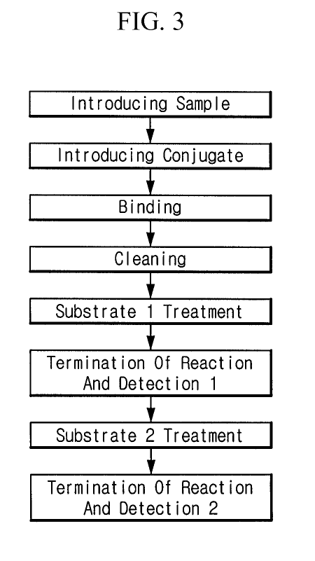

FIG. 3 is a flowchart schematically illustrating a process of detecting at least two types of target materials according to an embodiment. In the flowchart of FIG. 3, materials (i.e., conjugates), which specifically bind to at least two types of target materials and are labeled with an enzyme, are used as a detector probe. First, capture materials (e.g., antigens), which respectively recognize the at least two types of target materials (e.g., antibodies), specifically react with the target materials, and do not exhibit cross reactivity, are immobilized onto inner surface of the reaction chamber. In this regard, the immobilized capture materials do not need to have a specific arrangement. The immobilization of the capture materials in the reaction chamber may be performed using a known method used to immobilize materials, for example, biological molecules, on a substrate. For example, the capture materials may be immobilized by introducing a reactive group, such as an amino group using an aminosilane compound, to a portion of or the entire internal surface of the reactor chamber, and coupling the reactive group with an activated material. The activation of the material may be performed by activating a carboxyl group of the material in the form of an ester or anhydride.

Then, a buffer solution, a probe solution including at least two types of detector probes marked with enzymes (conjugates in FIG. 3), and samples including at least two types of target materials may be filled in each of the chambers, and the fluids may be transported to the reaction chamber using a centrifugal force generated by the rotation of the disc rotation body. Accordingly, the at least two types of target materials in the samples transported to the reaction chamber and the materials, which are immobilized in the reaction chamber and specifically react with the target materials, contact with each other to start a coupling reaction. In this regard, since the immobilized capture materials do not exhibit cross reactivity with each other, each of the capture materials specifically binds to respective target material to which it has selective (or specific) reactivity (see FIG. 2). Then, the reaction chamber is cleaned to remove unbound materials.

Then, a substrate 1 (e.g., 3,3′,5,5′-tetramethylbenzidine (TMB)) of a first enzyme (e.g., HRP) is added to the reaction chamber to convert the first substrate into a first chromogenic material by the action of the first enzyme in a complex including a probe material specifically binding to the first target material and conjugated with the first enzyme, the first target material, and a capture material immobilized in the reaction chamber and specifically binding to the first target material. The reaction is terminated, and absorbance of the first chromogenic material is measured at a particular wavelength to detect the target materials. The first chromogenic material of the reactants may be detected in the reaction chamber or in the detection chamber after being transported from the reaction chamber to the detection chamber. Then, a substrate 2 (p-nitrophenyl phosphate (PNPP)) of a second enzyme (e.g., AP) is added to the reaction chamber to convert the substrate 2 into a second chromogenic material using catalysis of the second enzyme in a complex including a probe material specifically binding to the second target material and conjugated with the second enzyme, the second target material, and a capture material immobilized in the reaction chamber and specifically binding to the second target material. Then, the reaction is terminated, and absorbance of the second chromogenic material is measured to detect the target material. The second chromogenic material may be detected in the reaction chamber or in the detection chamber after being transported from the reaction chamber to the detection chamber.

In this process, the probe solutions including the at least two types of detector probes, which respectively react with the respective target materials, are sequentially processed. Since the markers conjugated with each of the detector probes are different from each other, multiple target materials may be detected by identifying the markers.

In particular, the first probe solution is processed in a first detection chamber to detect a first target material combined with the first detector probe, an inlet of the first detection chamber is closed using a valve for opening and closing the chambers, and a second probe solution is processed in a second detection chamber to detect a second target material combined with the second detector probe. That is, each of the marker enzymes are sequentially subjected to reactions with each of substrates specifically binding to the marker enzymes in a single reaction chamber, and thus different results may be obtained.

Thus, a one-step detection and assay for detecting various target materials may be performed by attaching multiple antibodies, antigens, proteins, and genetic materials to a single reaction chamber, and specifically binding each of the target materials to each of biomolecules used to detect the target materials.

According to an embodiment, there is provided a method of controlling internal quality using the microfluidic device. The method may include: immobilizing capture materials, which each specifically react with a first target material and a second target material, respectively, in a reaction chamber; adding the first target material and the second target material to the reaction chamber, wherein the concentration of the second target material is known; detecting the first target material and the second target material by adding a solution including detector probes, which each specifically react with the first target material and the second target material, respectively, and are combined with markers, to the reaction chamber so that the detector probes are respectively combined with the first target material and the second target material, and measuring signals from the marker; and evaluating the degree of performance of the operations based on the detection results.

The terminologies are described above.

The terminology “internal quality control” is a method of controlling primary variations of tests, diagnoses, etc. to evaluate precision and accuracy of the results of experiments.

The method of controlling internal quality may include immobilizing capture materials, which each specifically react with the first target material and the second target material, respectively, in a reaction chamber. The immobilization of the materials in the reaction chamber may be performed using a known method used to immobilize materials, for example, biological molecules, on a substrate. For example, the materials may be immobilized by introducing a reactive group, such as an amino group using an aminosilane compound, to a portion of or the entire internal surface of the reactor chamber, and coupling the reactive group with the activated material. The activation of the material may be performed by activating a carboxyl group of the material in the form of an ester or anhydride. The first target material may be a subject to be assayed and contained in a sample. For example, the first target material may be selected from the group consisting of a protein, an antigen, an antibody, an enzyme, DNA, PNA, RNA, a hormone and a chemical material. At least two types of materials which specifically react with the first target material may be immobilized. The second target material may be a material having a known concentration or a known material having a high binding affinity with a material specifically binding to the second target material (e.g., having a nanomolar dissociation constant or less than nanomolar). Thus, the second target material may be used as an internal standard material. For example, the second target material and a material specifically binding to the second target material may be a pair of streptavidin and biotin. The second target material and the material specifically combined thereto may be selected from the group consisting of protein, nucleic acid, sugar, and chemical material, but are not limited thereto.

The method of controlling internal quality may include adding the first target material and the second target material to the reaction chamber. The second target material may be a material having a known concentration or a known material having a high binding affinity with a material specifically binding to the second target material (e.g., having a nanomolar dissociation constant or less than nanomolar).

The method of controlling internal quality may include detecting the first target material and the second target material by adding a solution including detector probes, which respectively specifically react with the first target material and the second target material and are combined with markers, to the reaction chamber so that the detector probes respectively bind to the first target material and the second target material. This detection process is described above in relation to one or more embodiments.

The method of controlling internal quality may include evaluating the degree of performance of the operations (performance quality) based on the detection results. For example, the method may further include determining the process as a reliable process when the results of the detection have a relation with the existence or concentration of the second target material, and determining the process as an unreliable process when the results of the detection do not have a relation with the existence or concentration of the second target material.

Thus, the internal quality control is a method of using a standard material as a control, by attaching materials (e.g., protein) specifically binding to different target materials to the reaction chamber, that is, attaching materials specifically binding to both materials to be detected and standard materials. In particular, a protein-protein interaction may be used.

The terminology “protein-protein interaction” is an interaction between proteins by a noncovalent force. The same or different polypeptide chains are combined with each other to express physiological functions. In a wider sense, antigen-antibody reactions or enzyme-substrate reactions may be regarded as the protein-protein interaction. That is, activation may be controlled by a subunit protein-protein interaction in subunit enzymes.

As described above, since each of the binding reactions of the first target material (e.g., antibody) and the second target material (e.g., streptavidin) does not affect each other, the binding reaction of the second target material may be performed at a constant level and function as a control regardless of the concentration of the first target material.

According to another embodiment, there is provided a method of controlling internal quality using the microfluidic device. The method may include: immobilizing capture materials, which respectively specifically react with a first target material and a second target material, in a reaction chamber; adding the first target material and the second target material to the reaction chamber, wherein the concentration of the second target material is known and the second target material is combined with a marker; detecting the first target material by adding a solution including a detector probe, which specifically reacts with the first target material and is combined with a marker, to the reaction chamber so that the detector probe is combined with the first target material, and measuring signals from the marker; and evaluating the degree of performance of the operations based on the detection results.

According to the above-described methods, various biological assays, chemical assays, immunoassays for hepatitis B, hepatitis C, rheumatoid, cancer, or the like, and genetic assays using DNA analysis may be performed using a single device. In addition, the disc may be efficiently designed by using a single reaction chamber, and thus the internal space, which are required for the reactions and/or detections, of the disc may be reduced, and the costs for manufacturing the disc may also be reduced due to the simplified process.

### EXAMPLE 1

Assay of Two Types of Target Materials Using Two Types of Antigen-Antibody Reaction

Prostate specific antigen (PSA) and human IgG were used as a first and a second target materials. An anti-PSA antibody and an anti-IgG antibody, which are antibodies of the above two target materials, respectively, were immobilized on the internal surface of a reaction chamber.

100 μl of each of the anti-PSA antibody and anti-IgG antibody in a coating buffer (50 mM Carbonate-bicarbonate buffer, pH 9.6) to a concentration of 830 ng/100 μl was introduced into a reaction chamber, and incubated at 4° C. overnight. After the reaction was terminated, 100 μl of the coating solution was removed, and 200 μl of a blocking buffer (10 mM phosphate, 0.14M NaCl, 1% BSA, pH7.4) was introduced into the reaction chamber in order to reduce non-specific bindings. After maintaining the resultant at 37° C. for 2 hours, the blocking buffer was removed.

Then, a target material PSA, which is capable of binding to the anti-PSA antibody (various concentrations) and another target material IgG, which is capable of binding to the anti-IgG antibody (constant concentration of 100 ng/chamber) were added to the reaction chamber. In this regard, an anti-PSA antibody conjugated with an HRP enzyme (having an antigen site, i.e., specificity, different from that of the anti-PSA antibody immobilized in the reaction chamber), which is capable of binding to the target material PSA and an anti-IgG antibody conjugated with an AP enzyme (having an antigen site, i.e., specificity, different from that of the anti-IgG antibody immobilized in the reaction chamber), which is capable of binding to the target material IgQ were added to the reaction chamber with the target materials.

Then, unbound materials were removed using a cleaning buffer (10 mM Phosphate, 0.14 M NaCl, 0.05% Tween-20, pH7.4), and a solution including TMB, which is a substrate of the HRP enzyme, was added to the reaction chamber. The reaction chamber was incubated for a particular time period, and the reaction was terminated. The reaction mixture was transported to the detection chamber to detect the first target material PSA. The PSA was detected by measuring absorbance at 450 nm according to each concentration.

Then, a solution including PNPP, which is a substrate of the AP enzyme, was added to the reaction chamber. The reaction chamber was incubated for a particular time period, and the reaction was terminated. The reaction mixture was transported to the detection chamber to detect the second target material IgG The IgG was detected by measuring absorbance at 405 nm.

The target material PSA and the anti-PSA antibody coated in the reaction chamber were used as a control.

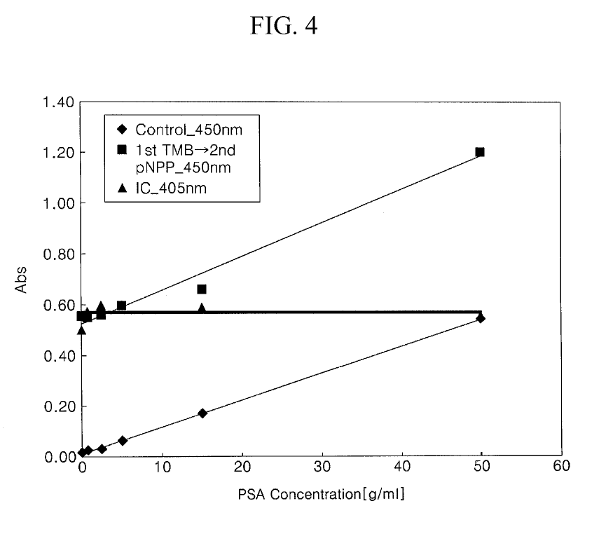

As a result, as shown in FIG. 4, a separate standard curve was obtained by using two types of antibodies coated in the reaction chamber, when compared with the existing method using a single antibody. In addition, it was identified that signals were constantly measured regardless of the concentration of the target antibody sample. Thus, it was identified that each of the antibodies may be detected by performing immunoassay in which each of the capture antibodies reacts to different target antibodies individually and one of the antibodies may be used as an internal control. In FIG. 4, the square indicates the sum of the results obtained by detecting the first target material PSA by measuring fluorescent absorbance in a first detection chamber at 450 nm and the results obtained by detecting the second target material IgG by measuring absorbance in a second detection chamber at 450 nm. The triangle indicates the results of the control measured at 405 nm. These results were obtained using a certain concentration and indicate that a plurality of assays may be performed using various antibodies and, at the same time, absorbance according to the concentrations may be measured. The diamond in FIG. 4, as a control, indicates the results obtained by using immobilized anti-PSA in the reaction chamber, binding PSA to an anti-PSA, binding an anti-PSA conjugated with an HRP, adding TMB, substrate of the HRP thereto, incubating the mixture, terminating the reaction, and measuring absorbance at 450 nm.

### EXAMPLE 2

Internal Quality Control Using Streptavidin-Biotin Interaction

First, anti-PSA specifically reacts with a first target material PSA to be detected, and streptavidin which is a protein having a high affinity to biotin, the second target material, and selected for the internal quality control, were coated on the internal surface of the reaction chamber. The coating was performed in the same manner as in Example 1.

Then, samples of a patient (according to the concentrations of PSA) and biotin conjugated with AP were simultaneously added to the reaction chamber to detect the first target material PSA. Accordingly, the PSA bound to the anti-PSA antibody, and the AP-biotin bound to the streptavidin.

As in Example 1, unbound materials were removed using a cleaning buffer (10 mM Phosphate, 0.14 M NaCl, 0.05% Tween-20, pH7.4), and a solution including TMB, which is a substrate of the HRP enzyme, was added to the reaction chamber. The reaction chamber was incubated for a particular time period, and the reaction was terminated. The reaction mixture was transported to the detection chamber to detect the first target material PSA. The PSA was detected by measuring absorbance at 450 nm according to the concentrations. Then, a solution including PNPP, which is a substrate of the AP enzyme, was added to the reaction chamber. The reaction chamber was incubated for a particular time period, and the reaction was terminated. The reaction mixture was transported to the detection chamber to detect the second target material biotin. The biotin was detected by measuring absorbance at 405 nm.

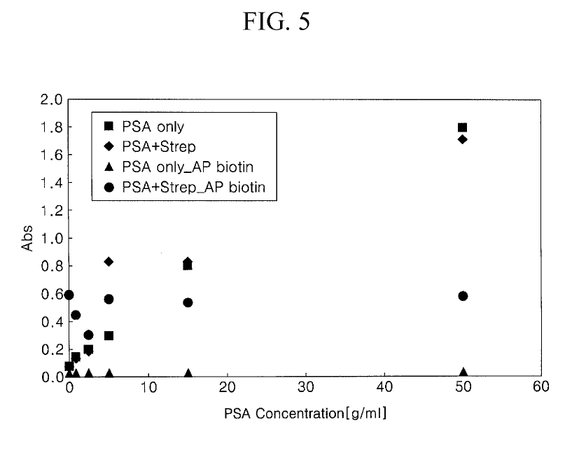

As a result, as shown in FIG. 5, it was identified that the specific bindings of the single target antibody and protein streptavidin do not affect each other. That is, an interaction between streptavidin and AP-biotin was maintained at a constant level regardless of the concentration of the target antibody (PSA). Thus, internal quality control (internal QC) may be performed using the interaction between streptavidin and AP-biotin as a control. In FIG. 5, the PSA only indicates the results of PSA detection when only anti-PSA is coated in the reaction chamber. The PSA+Strep indicates the results of PSA detection when anti-PSA and streptavidin are coated in the reaction chamber. The PSA+Strep_Ap biotin indicates the results of Ap-biotin detection when anti-PSA and streptavidin are coated in the reaction chamber. The PSA only_Ap biotin indicates the results of Ap-biotin detection when only anti-PSA is coated in the reaction chamber.

Thus, multiple assays may be performed using a single reaction chamber by using the microfluidic structure according to an embodiment, and thus the internal space of a disc and the amount of the sample required for the assay may be reduced. In addition, since at least two types of materials may be detected in a single detection, at least two types of biological samples may be assayed, and internal quality control (internal QC) may be performed using the microfluidic structure as a control for the operations, thereby resulting in excellent industrial efficiency.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. A microfluidic structure comprising:
a plurality of sample chambers;
a reaction chamber in which at least two types of capture materials, which each specifically react with at least two types of target materials, respectively, are immobilized;
a detection chamber, in which a reaction of the at least two types of the capture material with the at least two types of target materials is detected, the detection chamber being connected to the reaction chamber;
a path connecting the chambers; and
a valve for opening and closing the path.

2. The microfluidic structure of claim 1, wherein each of the at least two types of capture materials is a different material from the others of the at least two types of the capture materials.

3. The microfluidic structure of claim 1, wherein the target materials and the capture materials are selected from a group consisting of a protein, an antigen, an antibody, an enzyme, deoxyribonucleic acid (DNA), peptide nucleic acid (PNA), ribonucleic acid (RNA), a hormone, and a chemical material.

4. The microfluidic structure of claim 3, wherein the target materials and the capture materials are selected from a group consisting of an antigen, an antibody, and a protein.

5. The microfluidic structure of claim 1, wherein the plurality of sample chambers comprises at least one chamber selected from a group consisting of a buffer solution chamber, a substrate solution chamber, a probe solution chamber, and a biological sample chamber comprising the at least two types of target materials.

6. The microfluidic structure of claim 5, wherein the probe solution comprises at least two types of detector probes which each respectively specifically react with each of the at least two types of target materials.

7. The microfluidic structure of claim 6, wherein the at least two types of detector probes are respectively conjugated with a marker, wherein the marker for a respective detector probe is different from other markers for the other detector probe.

8. The microfluidic structure of claim 7, wherein the marker is one selected from a group consisting of an enzyme, a fluorescent material, a radioactive isotope, and a chemical material.

9. A microfluidic device based on a centrifugal force comprising a rotation body and a microfluidic structure according to claim 1, wherein a fluid in the microfluidic structure is transported using the centrifugal force generated by the rotation of the rotation body.

10. A method of assaying at least two types of target materials using a microfluidic device,
wherein the microfluidic device is based on a centrifugal force and comprises a rotation body and a microfluidic structure, wherein a fluid in the microfluidic structure is transported using the centrifugal force generated by the rotation of the rotation body, the microfluidic structure comprising:
a plurality of sample chambers housing a sample fluid which comprises at least two types of target materials to be detected;
a reaction chamber in which at least two types of capture materials, which each specifically react with the at least two types of target materials, respectively, are immobilized;
a detection chamber, in which a reaction of the at least two types of the capture material with the at least two types of target materials is detected, the detection chamber being connected to the reaction chamber;
a path connecting the chambers; and
a valve for opening and closing the path,
the method comprising:
introducing the sample comprising the at least two types of target materials to the reaction chamber so that the at least two types of target materials contact with the at least two types of capture materials; and
adding a solution comprising at least two types of detector probes, which each specifically react with the at least two types of target materials, respectively, and are conjugated with a marker, to the reaction chamber, so that the at least two types of target materials contact with the at least two types of detector probes.

11. The method of claim 10, further comprising detecting the target materials by measuring signals from the marker conjugated with the detector probes.

12. The method of claim 11, wherein the detecting is performed in the reaction chamber or in the detection chamber to which the reaction solution is transported from the reaction chamber.

13. The method of claim 12, wherein the marker is an enzyme and the detecting is performed by adding a substrate, which is converted by the enzyme into a chromogenic material which absorbs light at a particular wavelength, to the reaction chamber to form the chromogenic material, and measuring signals from the chromogenic material.

14. The method of claim 13, comprising:
adding a first substrate, which is converted into a first chromogenic material by a first enzyme, to the reaction chamber where the first substrate is converted to the first chromogenic material, transporting the first chromogenic material to a first detection chamber, and measuring a first signal from the first chromogenic material; and
adding a second substrate, which is converted into a second chromogenic material by a second enzyme, to the reaction chamber where the second substrate is converted to the second chromogenic material, transporting the second chromogenic material to a second detection chamber, and measuring a second signal from the second chromogenic material.

15. The method of claim 10, wherein the sample and the solution comprising the detector probes are simultaneously added to the reaction chamber.

16. The method of claim 10, wherein the adding of the solution comprising the at least two types of detector probes to the reaction chamber is performed by sequentially or simultaneously adding a solution comprising a first detector probe and a solution comprising a second detector probe to the reaction chamber.

17. A method of controlling internal quality using a microfluidic device, wherein the microfluidic device is based on a centrifugal force and comprises a rotation body and a microfluidic structure, wherein a fluid in the microfluidic structure is transported using the centrifugal force generated by the rotation of the rotation body,
the microfluidic structure comprising:
a plurality of sample chambers housing a sample which comprises a first target material and a second target material to be detected;
a reaction chamber to receive the sample and where the first and the second target materials are to be contacted with a first and a second capture materials, which specifically react with the first and the second target materials, respectively;
a detection chamber, where reactions of the first and the second capture material with the first and the second target materials are detected, the detection chamber being connected to the reaction chamber;
a path connecting the chambers; and
a valve for opening and closing the path,
the method comprising:
immobilizing the capture materials in the reaction chamber;
adding the first target material and the second target material to the reaction chamber, wherein a concentration of the second target material is known;
detecting the first target material and the second target material by adding a solution comprising detector probes, which each specifically react with the first target material and the second target material, respectively, and are conjugated with a marker, to the reaction chamber so that the detector probes each bind to the first target material and the second target material, respectively, and measuring signals from the marker; and
evaluating the degree of performance of the operations based on the detection results.

18. The method of claim 17, wherein the assay results of the second target material is used as a control.

19. A method of controlling internal quality using a microfluidic device,
wherein the microfluidic device is based on a centrifugal force and comprises a rotation body and a microfluidic structure, wherein a fluid in the microfluidic structure is transported using the centrifugal force generated by the rotation of the rotation body, the microfluidic structure comprising:
a plurality of sample chambers housing a sample which comprises a first target material and a second target material to be detected;
a reaction chamber to receive the sample and where the first and the second target materials are to be contacted with a first and a second capture materials, which specifically react with the first and the second target materials, respectively;
a detection chamber, where reactions of the first and the second capture material with the first and the second target materials are detected, the detection chamber being connected to the reaction chamber;
a path connecting the chambers; and
a valve for opening and closing the path,
the method comprising:
immobilizing the capture materials in the reaction chamber;
adding the first target material and the second target material to the reaction chamber, wherein a concentration of the second target material is known and the second target material is conjugated with a marker;
detecting the first target material by adding a solution comprising a detector probe, which specifically reacts with the first target material and is conjugated with a marker, to the reaction chamber so that the detector probe binds to the first target material, and measuring signals from the marker; and
evaluating the degree of performance of the operations based on the detection results.

20. A method assaying at least two types of target materials using a microfluidic device,
wherein the microfluidic device is based on a centrifugal force and comprises a rotation body and a microfluidic structure, wherein a fluid in the microfluidic structure is transported using the centrifugal force generated by the rotation of the rotation body, the microfluidic structure comprising:
a plurality of sample chambers housing a sample fluid which comprises at least two types of target materials to be detected;
a reaction chamber in which at least two types of capture materials, which each specifically react with the at least two types of target materials, respectively, are immobilized;
a detection chamber, in which a reaction of the at least two types of the capture material with the at least two types of target materials is detected, the detection chamber being connected to the reaction chamber;
a path connecting the chambers; and
a valve for opening and closing the path,
the method comprising:
adding a sample comprising at least two types of target materials to a reaction chamber so that the at least two types of target materials contact with the capture materials, wherein if the target material is nucleic acid, the nucleic acid is conjugated with a marker; and
adding a solution comprising at least two types of detector probes, which respectively specifically react with one of the at least two types of target materials and are conjugated with a marker, so that the at least two types of target materials contact with the at least two types of detector probes.
